(19) United States
(12) Patent Application Publication
(10) Pub. No.: US 2005/0075636 A1
(43) Pub. Date: Apr. 7, 2005

(54) Bone anchor

(76) Inventor: Leo Gotzen, Marburg (DE)

Correspondence Address: GIFFORD, KRASS, GROH, SPRINKLE & CITKOWSKI, P.C, PO BOX 7021, TROY, MI, 48007-7021, US

(21) Appl. No.: 10/499,399

(22) PCT Filed: Nov. 11, 2002
(86) PCT No.: PCT/EP02/12588

(51) Int. Cl. A61B 17/58

(52) U.S. Cl. 606/72

(57) ABSTRACT

The invention relates to a bone anchor for re-fixing soft tissue to bones, comprising a base body, which is made of bone, and comprising two through holes provided for accommodating a suture.

The present invention relates to a bone anchor for the refixing of soft tissue to bones with the help of a suture.

Such bone anchors have previously only been known in metal or plastics and serve, after a rupture of soft tissues (ligaments, tendons and muscles) in the region of the joints, above all to achieve a refixation of the soft tissue to bones. For this purpose, the anchors are lowered into a bore of the bone, with a suture being fixed to the anchor and with the torn off soft tissue being able to be fixed to the bone again by it.

It is the object of the present invention to provide a bone anchor which is particularly compatible and which can be particularly firmly anchored in a bone.

This object is satisfied by the features of claim 1 and in particular in that the bone anchor consists of a substantially cylindrical body made in one piece from human or animal cortical bone which has at least two passage bores with two openings each, with one opening of one bore being arranged adjacent to an opening of the other bore.

The bone anchor in accordance with the invention is characterized, on the one hand, by excellent biocompatibility and osteointegration, since the bone anchor is made from natural bone material which can grow together with the surrounding bone material of the patient particularly well without disturbances arising hereby. At the same time, the bone anchor is very stable due to the cortical or compact bone used, that is the bone anchor in accordance with the invention has just as large a security against tearing as comparable metal anchors.

Finally, the bone anchors in accordance with the invention can be fixed very firmly and securely into the bore of a surrounding bone in a particularly simple manner. For this purpose, the suture for the refixing of the soft tissue is led through the two bores of the bone anchor such that two free suture ends exit in each case the two adjacent openings of the two bores. It is possible in this manner, after introduction of the bone anchor into the bore of the patient's bone, to pull abruptly on the two free suture ends, whereupon a torque is applied to the bone anchor. If this is large enough, the bone anchor is lightly turned in the surrounding spongious bone of the patient and thereby canted. The anchor is securely anchored in this manner such that it no longer releases from the bore.

Advantageous embodiments of the invention are recited in the dependent claims, in the description and in the drawings.

In accordance with a first advantageous embodiment, the two adjacent openings can be arranged in the region of an edge of the body, e.g. a front edge. If the bone anchor is inserted into the associated bore with this edge to the front, the torque or tilting moment which arises when the suture ends threaded in the manner described above are abruptly pulled is particularly large.

In accordance with a further advantageous embodiment, the body can taper at its front end, with the two adjacent openings being able to be arranged in the region of the front end. In this embodiment, a particular simple and secure introduction of the bone anchor into the bore is ensured due to the tapering front end. At the same time, the arising tilting moment or torque is maximized.

It is advantageous for one bore to run transversely to the longitudinal axis of the body, since the weakening of the base body by the bore is minimized in this manner. It is equally advantageous for both bores to extend toward one another at an angle, since this likewise minimizes the weakening of the body, whereby the strength of the body is increased. A particularly stable bone anchor results when both bores extend toward one another at an angle of approximately 45°, since in this case a compromise is reached between the position and the spacing of the two bores.

It is furthermore advantageous for the extent of the suture and for the stability of the bone anchor for the two bores to lie substantially in one plane.

A groove can be provided at the outer extent of the body between two openings of two bores. It is ensured by such an outside groove that the suture threaded into the bone anchor is not damaged or does not tear on the introduction of the bone anchor into the associated bore, for example by means of a pressed fit. It is particularly advantageous in this process for that groove, into which the two adjacent openings open, extends up to the upper end face of the body, since an exit passage for the two suture ends is hereby created. It is equally advantageous for that groove, which connects the two other openings of the bores, to extend only between these two openings, since the body of the bone anchor is hereby weakened the least. For the same reason, the width of this groove can be minimized, whereas the width of the groove extending up to the rear end of the body is advantageously selected to be just as wide as the openings, since it is hereby ensured that the suture is not jammed between the outer wall of the bone anchor and the bore and thereby tears under certain circumstances on introduction of the bone anchor.

Peripheral grooves are preferably provided at the outer periphery of the body and extend concentrically to the longitudinal axis of the body. In this manner, the bone anchor can be introduced by means of a press fit into a bone bore which has a slightly lower diameter than the outer diameter of the anchor.

The present invention will be described in the following purely by way of example with reference to advantageous embodiments and to the enclosed drawings. There are shown:

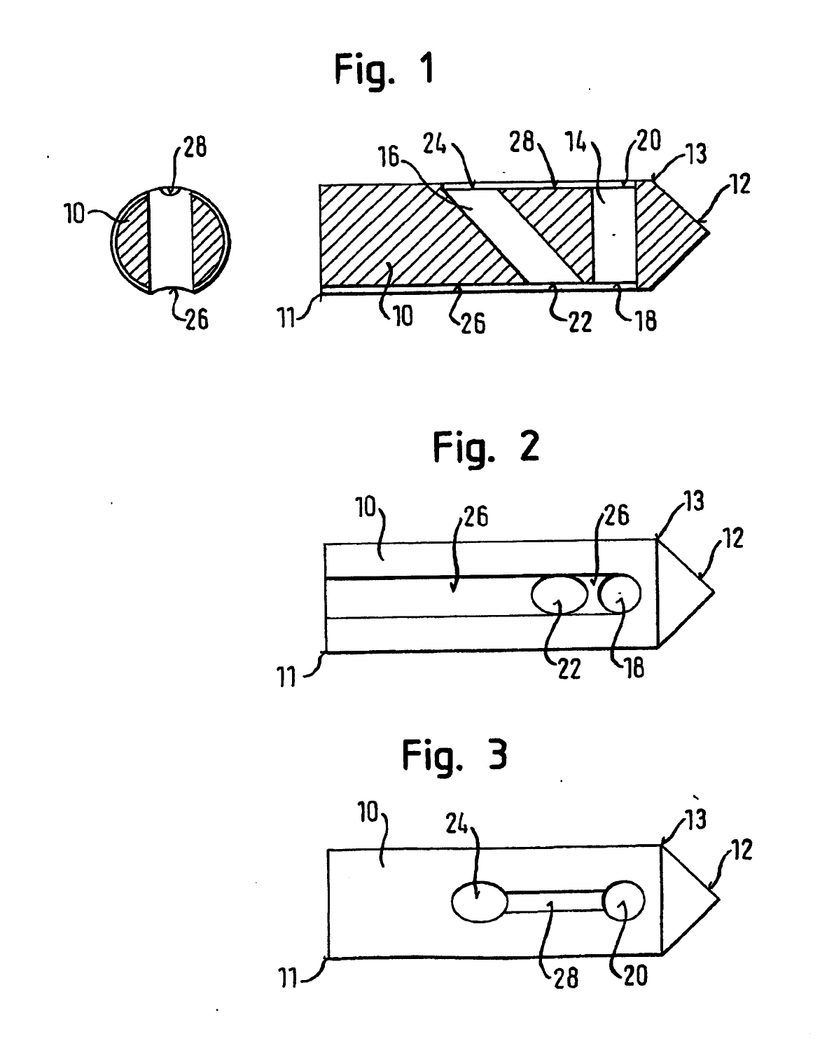

FIG. 1 a longitudinal section through a bone anchor and a cross-section through the bone anchor along the broken line;

FIG. 2 a side view of the bone anchor of FIG. 1;

FIG. 3 a further side view of the bone anchor of FIGS. 1 and 2;

FIG. 4 a further embodiment of a bone anchor which is provided with a suture and introduced into a bone bore; and

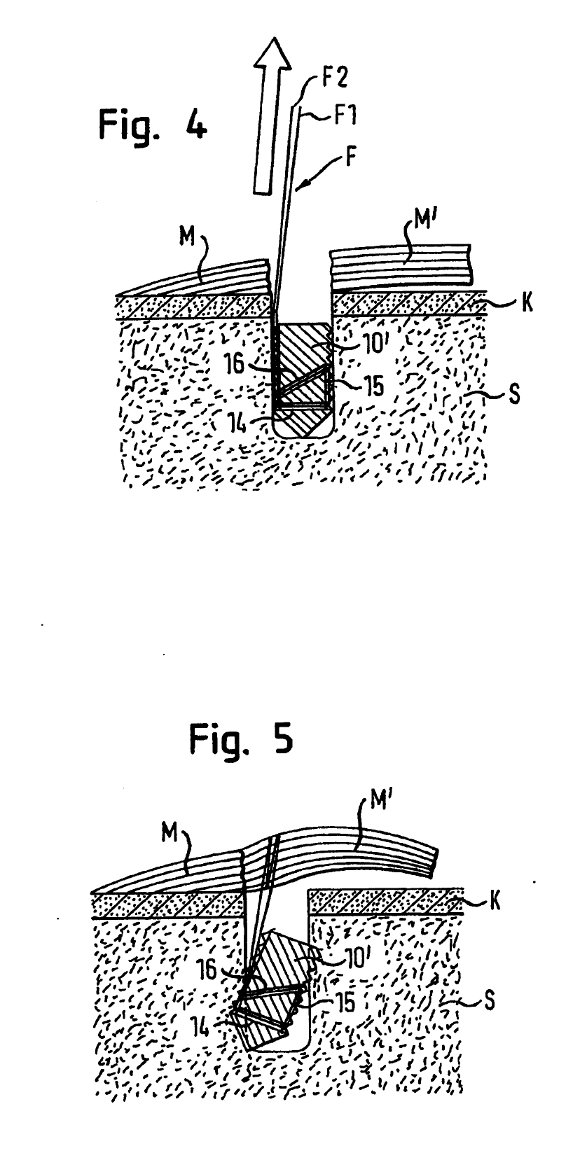

FIG. 5 the bone anchor of FIG. 4 in the completely anchored state.

The bone anchor shown in FIGS. 1 to 3 consists of a substantially cylindrical body 10 made in one piece of preserved human or animal cortical bone. The body is elongate and circularly cylindrical, with the front end 12 of the body tapering conically. The acute angle to the longitudinal axis of the body amounts to approximately 45° here. The transition between the circularly cylindrical section and the conical tip of the body defines an edge 13.

The body 10 has two passage bores 14 and 16, with the passage bore 14 having two openings 18 and 20 and the bore 16 having two openings 22 and 24. Both bores 14 and 16 have the same diameter. The bore 14 extends at right angles to the longitudinal axis of the body 10. The bore 16 extends at an angle of approximately 45° to the longitudinal axis of the bore 14, with both bores 14 and 16 lying in the same plane.

As in particular FIG. 2 shows, the opening 18 of the bore 14 at the front end of the body 10 is adjacent to the opening 22 of the bore 16. Both openings 18 and 22 are only spaced a few millimeters apart from one another and lie close to the front edge 13.

FIG. 3 shows that, due to the oblique orientations of the two bores 14 and 16 toward one another, the two other openings 20 and 24 are clearly spaced apart from one another, that is the opening 20 likewise lies in the region of the front end of the body 10, whereas the bore 24 lies approximately in the region of the upper third of the body 10.

FIGS. 1 to 3 furthermore show that two openings 18 and 22 and 20 and 24 respectively are connected by a groove with a concave cross-section provided at the outer periphery of the body 10. FIG. 2 shows that the two adjacent openings 18 and 22 are connected to one another by a groove 26 which extends up to the rear end or to the rear edge 11 of the body 10. In contrast, FIG. 3 shows that the two openings 20 and 24 are connected to one another by a groove 28 which only extends between the two openings 20 and 24. Both grooves 26 and 28 extend at the outer periphery of the body 10 parallel to its longitudinal axis, with the width of the groove 26 corresponding to the diameter of the bores 14, 16 and the width of the groove 26 amounting to approximately 50% of the diameter of the bores 14, 16.

The bone anchor shown in FIGS. 1 to 3 can have a length of, for example, approximately 8 to 16 mm and a diameter of, for example, approximately 2 to 5 mm. The diameter of the bores can vary, for example, between approximately 0.8 and 1.5 mm. The width of the grooves can vary between approximately 0.5 and 1.6 mm. Different base dimensions are naturally also possible here. It is in every case advantageous for the body of the bone anchor to be substantially elongate and hollow cylindrical.

FIGS. 4 and 5 show a further embodiment of a bone anchor 10′ which, however, only differs from the embodiment shown in FIGS. 1 to 3 in that a plurality of peripheral grooves 15 concentric to the longitudinal axis are provided at the outer periphery of the body 10′ and promote a fixing of the bone anchor in the associated bore by means of a press fit.

As FIGS. 4 and 5 show, a suture F is guided through the bores 14 and 16 such that the two free suture ends F1 and F2 each exit through the two adjacent openings 18 and 22. For this purpose, one suture end of the suture F is guided into the opening 18, then through the bore 14 out of the opening 20, is subsequently guided through the opening 24 into the bore 16 and, finally, through the opening 22 out of the bore 16 such that the two free suture ends F1 and F2 can be guided in the groove 26 to the rear end of the bone anchor in the region of the edge 11 of the body 10′.

FIGS. 4 and 5 show the cross-section through a human bone with a cortex K and spongiosa S. A muscle insertion M, from which a piece M′ has been torn, is located above the cortex.

To fix the bone anchor 10′, one bore is introduced into the bone which is so deep that the bone anchor 10′ lies below the cortex when it is completely lowered into the borehole. The borehole is selected to be slightly smaller than the cross-section of the bone anchor 10′ such that the bone anchor 10′, as shown in FIG. 4, can be anchored in the borehole by means of a press fit. The two free suture ends F1 and F2 of the suture F are guided in the groove 26 in this process such that the suture cannot be squeezed between the bone anchor 10′ and the surrounding borehole.

After the bone anchor has been introduced into the bore of the bone, as shown in FIG. 4, the two suture ends F1 and F2 are pulled at the same time with a strong jerk in the direction of the arrow drawn in FIG. 4. A torque or a tilting moment is hereby applied to the bone anchor 10′ so that, as shown in FIG. 5, it rotates in the spongiosa S and is canted therein. The bone anchor 10′ is hereby securely anchored and secured against being torn out. The torn off piece of muscle M′ can subsequently be sewn tight with the help of the suture F so that it can again grow together with the bone.

The bone anchor in accordance with the invention can be sterilized by autoclave or by gamma radiation. Sterilization by application of damp heat in an order of magnitude of 120° C. for a duration of 20 minutes and by subsequent drying to 60 to 50° C. within 30 minutes has proved to be advantageous.

### Reference Numeral List

10, 10′ body
11 edge
12 front end
13 edge
14 bore
15 grooves
16 bore
18, 20 opening
22, 24 opening
26, 28 groove
F suture
F1, F2 suture ends
K, cortex
M muscle tissue
M′ torn-off piece
S spongiosa

1. A bone anchor for the refixing of soft tissue to bone with the help of a suture, comprising a cylindrical body having an edge, a front end, an opposing end face, a periphery, and a longitudinal axis made in one piece from human or animal cortical bone whose front end tapers conically and which has at least two passage bores, each of the at least two passage bores having two opposing openings, wherein:
the at least two passage bores lie in one plane and extend toward one another at an angle;
a first opening having a first width in one of the at least two passage bores is arranged adjacent to a second opening having a second width of another of the at least two passage bores;
an opening opposing the first opening and having an opposing first opening width in one bore is arranged spaced apart from an opening opposing the second opening and having an opposing second opening width of another bore; and
the first opening and the second opening are arranged proximal to the front end.

2. A bone anchor in accordance with claim 1, characterized in that the first opening and the second opening are proximal to an edge of the body.

3. A bone anchor in accordance with claim 1, characterized in that one of the at least two passage bores extends transversely to the longitudinal axis of the body.

4. A bone anchor in accordance with claim 1, characterized in that one of the at least two passage bores extend at an angle, in particular an angle of approximately 45°, to another of the at least two passage bores.

5. A bone anchor in accordance with claim 1, further comprising a groove at the periphery of the body between the at least two passage bores.

6. A bone anchor in accordance with claim 1, further comprising a groove at the periphery of the body which extends parallel to the longitudinal axis of the body and extends between the first opening and the second opening and up to an end face of the body.

7. A bone anchor in accordance with claim 1, further comprising a groove at the periphery of the body which extends parallel to the longitudinal axis of the body and having a width that corresponds to the width of one of the first opening or the second opening.

8. A bone anchor in accordance with claim 1, further comprising a groove at the periphery of the body which extends parallel to the longitudinal axis of the body and having a width that is smaller than the width of one of the first opposing opening and the second opposing opening.

9. A bone anchor in accordance with claim 1, further comprising a plurality of peripheral grooves in the periphery of the body which extend concentrically about the longitudinal axis of the body.

10. A bone anchor in accordance with claim 1, characterized in that the suture has a first free suture end guided through the at least two passage bores and a second free suture end such that the first free suture end exits the first opening and the second free suture end exits the second opening at the periphery of the body.
